(19) United States
(12) Plant Patent Application Publication
(10) Pub. No.: US 2002/0002725 P1
(43) Pub. Date: Jan. 3, 2002

(54) Gladiolus plant named 'Festiva'

(76) Inventor: John O. Zipperer III, Ft. Myers, FL (US)

Correspondence Address: Laura G. Barrow, Esq., P.O. Box 215, Estero, FL, 33928-0215, US

(21) Appl. No.: 09/877,575

(22) Filed: Jun. 8, 2001

(30) Foreign Application Priority Data
Jun. 9, 2000 (EP) 2000/0892

(51) Int. Cl. A01H 5/00

(52) U.S. Cl. PLT/301

(57) ABSTRACT

A new and distinct gladiolus variety, designated ‘Festiva’, is shown and described. Compared to the ‘Spic and Span’ variety, the ‘Festiva’ variety is 15 cm taller, has 2 to 3 more florets, a larger bloom diameter, and a similar resistance to foliar diseases.

The present invention comprises a new and distinct variety of a Gladiolus plant referred to by the variety name ‘Festiva.’

### BRIEF DESCRIPTION OF THE PHOTOGRAPHS

FIG. 1 is a photograph of a ‘Festiva’ variety plant in bloom.

FIGS. 2 and 3 are photographs of the ‘Festiva’ variety plant prior to blooming.

### ASEXUAL REPRODUCTION

The new variety was originated by the Applicant in a controlled proprietary breeding program in Ft. Myers, Fla. wherein selected gladiolus varieties were crossed. (The provisional breeder's designation “28-3” was used in a related application for a Community plant variety right filed earlier in the European Union Community Plant Variety Office). The female parent was a gladiolus variety named ‘Dr. Magee,’ characterized in part by having a small pink bloom, a short stem having a short flower head, and high resistance to Fusarium fungi species. The male parent was a salmon pink gladiolus variety named ‘Jennie Lee,’ an unpatented, released variety (by Jennie Lee Zipperer), characterized in part by having a long stem, long head, and high floret count (i.e. 18-20 florets). The seeds were planted in Ft. Myers, Fla., and the selection of ‘Festiva’ variety was made in Spring 1988. Asexual reproduction of the ‘Festiva’ variety was achieved by collecting cormels from the first corm. All subsequent asexual reproductions of the ‘Festiva’ variety remain true to the original variety type.

### BOTANICAL DESCRIPTION

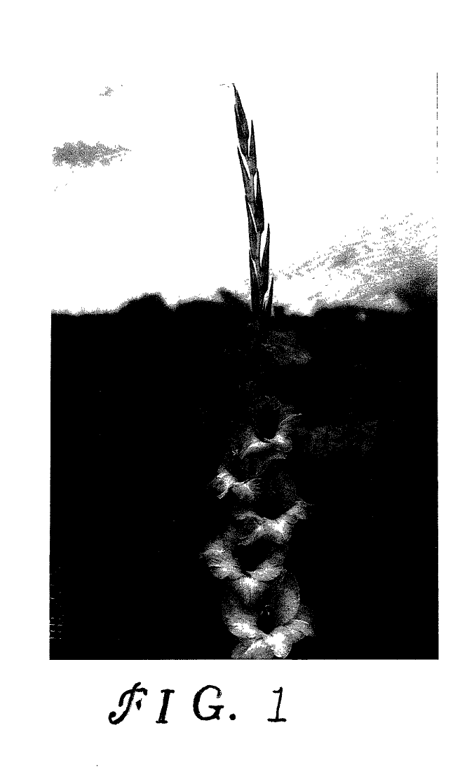

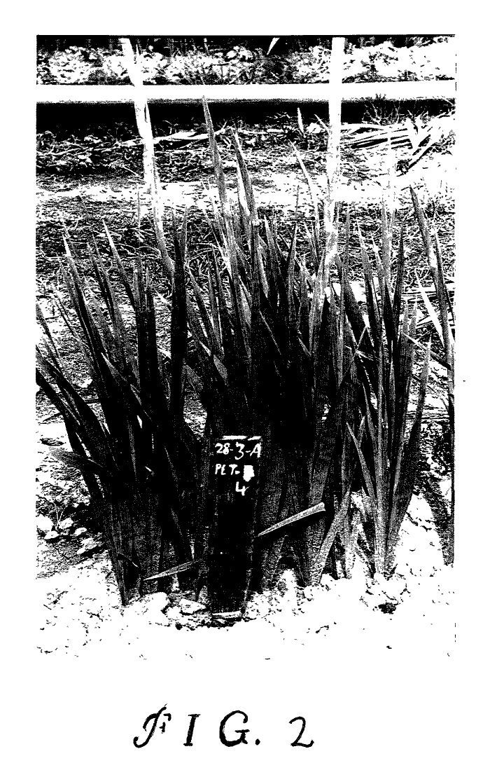

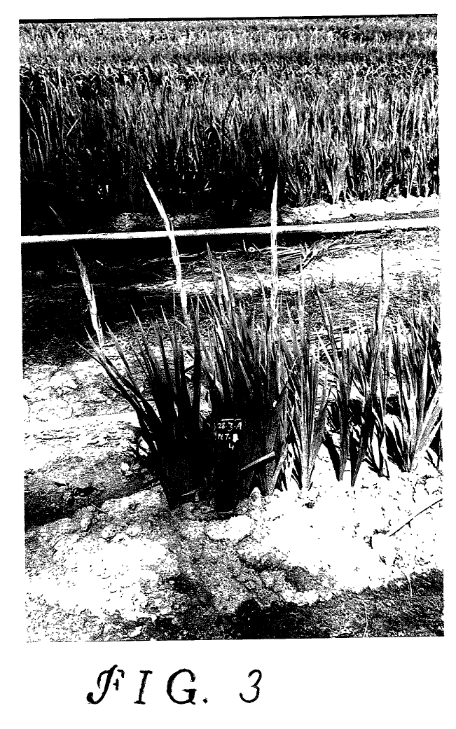

The accompanying color photograph (FIG. 1) shows the inflorescence and various stages of blooming of the ‘Festiva’ variety plant.

The bud size of the ‘Festiva’ variety is about 7.5 cm in length. The flowers of the variety each consist of a total of six petals. Specifically, each bloom consists of three large petals oriented on the top part of the bloom, one medium-size petal oriented on the lower half of the bloom (at about 6:00), and 2 smaller-size petals also oriented on the lower part of the bloom. Instead of one medium- and two small-size petals on the lower half of the bloom, some spikes of the present variety exhibit two medium- and one smallsize petal on the lower half of the bloom. The orientation of three upper large petals as well as the color and distinctive markings on all six petals are consistent, however, among subsequent asexual reproductions. The diameter of the entire bloom is about 13 cm.

The color of the corolla bloom is purple/red deep within the throat of the bloom, followed by a pink orange color mid-throat with a light pink color extending throughout the rest of the bloom. The medium-size petal(s) has a splash of white color in the center and each of the smaller petals has a splash of yellow in the center. All six petals have a white line bisecting the petal lengthwise.

The spike of the plant is about 118 cm and comprises 17 florets when grown from #2 size corms (1.25 to 1.50 inches) in Ecuador. The head length is 48 cm. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are white, and the stamens are purple/red.

The leaf color of the ‘Festiva’ variety is green. The leaf size is about 63 cm (length) by 3.5 cm (width).

### GENERAL OBSERVATIONS

Compared to the gladiolus variety ‘Spic and Span,’ the ‘Festiva’ variety is about 15 cm taller, has 2 to 3 more florets, has a bloom diameter that is about 2 to 3 cm larger, and a similar resistance to foliar diseases (i.e. medium resistance to Fusarium and good resistance to Curvalaria). The time to harvest is similar to the ‘Advance’ variety (i.e. about 85 to 86 days). The ‘Festiva’ variety has performed well when grown during fall, winter, and spring months in Ecuador. Growth is good during short days as well as in cold weather. The variety shows good resistance to burning in hot weather.

The variety opens well after traveling up to seven days in a dry box, and can hold up to six florets open simultaneously in a vase of water.

I claim:

1. A new and distinct gladiolus variety ‘Festiva’, as shown and described herein.
